(19) United States
(12) Patent Application Publication
(10) Pub. No.: US 2023/0133658 A1
(43) Pub. Date: May 4, 2023

(54) A METHOD FOR PROCESSING RAW PLANT MATERIAL, ESPECIALLY LEGUMES INTO PTOTEIN HAVING A NUTRITIONAL AND FEED VALUE, BIOETHANOL, BIOGAS AND FERTILISER MATERIALS

(71) Applicant: BIOTECHNIKA TOMASZ KAPELA, LODZ (PL)

(72) Inventors: Tomasz KAPELA, LODZ (PL); Krzysztof MAKOWSKI, ZGIERZ (PL)

(21) Appl. No.: 17/917,932

(22) PCT Filed: Apr. 7, 2021
(86) PCT No.: PCT/PL2021/050022

§ 371 Date: Oct. 8, 2022

(30) Foreign Application Priority Data
Apr. 8, 2020 (PL) P.433480

(51) Int. Cl. C05F 5/00; C12P 5/02; C12P 7/08; A23J 1/14

(52) U.S. Cl. CPC C05F 5/008; A23J 1/148; C12P 5/023; C12P 7/08

(57) ABSTRACT

A method for processing raw plant material, especially legumes into protein having a nu-tritional and feed value, bioethanol, biogas and fertiliser materials characterised in that the raw plant material is subjected to a dehusking process, followed by the dehusked raw material being crushed and subjected to extraction with stirring, wherein the insoluble solid fraction is separated and subjected to enzymatic hydrolysis using liquefying and saccharifying enzymes, then subjected to ethanol fermentation, where the produced ethanol is distilled and the digestate is transferred as a substrate for biogas production, and the liquid fraction after the extraction process is subjected to a process of precipitation to a solid form in the form of protein precipitate, which is washed with extraction buffer, after which the liquid residue is removed and subjected to a biogasification process.

The invention relates to a method for processing raw plant material, especially legumes into protein having a nutritional and feed value, bioethanol, biogas and fertiliser materials.

Legumes have been known and cultivated by mankind for thousands of years. Their popularity and share in the diet has varied many times over this period. In countries of Central and South America and Asia as well as in some of the Middle East, plants such as peas, beans, soya beans and chickpeas are a diet staple, while in other parts of the world there an increased consumption of legumes is observed in addition to that of soya beans, which continue to rank first in terms of crops. Due to their composition, rich in protein and carbohydrates, they are an excellent component of the diet. However, unfortunately, they also have some disadvantages, such as the presence of sugars that are not digested by humans and anti-nutrients. These disadvantages do not disqualify legumes as food, but they do involve certain limitations.

Consumption of legumes is not constant and production volumes depend partly on dietary trends. One of the main trends affecting the increased farming of legumes is the elimination or reduction of gluten in human diet. As it is, legumes are virtually the only alternative to this protein obtained from cereals. Due to growing knowledge and awareness of nutrition, apart from reducing gluten in the diet, people also disfavour excessive amounts of carbohydrates, which are a group of compounds that are abundant in seeds of legumes and can represent as much as more than 50% of the dry weight of the seeds. It is therefore a very interesting challenge to obtain pure forms of protein from legumes while making use of the resulting by-product streams.

Document WO2005079190 discloses a process for providing conversion of biomass materials with a high level of solids, in particular grains of cereals and legumes, in order to obtain a conversion with a high level of solids to produce ethanol and high protein by-products, such as dried stillage with solutes, comprising: treating biomass material with an acid, heating the acid-treated biomass material at a temperature sufficient for pasting and solubilisation of starch and breakdown of carbohydrates other than starch and lignin, adding stillage, cooling the suspension to temperatures which allow the use of liquefying enzymes, adding saccharification enzymes to hydrolyse the starch and non-starch carbohydrates into respective sugars, cooling the hydrolysate and transferring it to an ethanol fermentor, concentrating the ethanol and obtaining the fraction containing the biomass residue with an addition of with protein from the dried stillage with solubles.

Document ATA56991 discloses a method of producing ethanol from protein-rich starch-containing raw materials, e.g. from legumes, wherein the raw materials are milled, the starch content undergoes enzymatic degradation or acid hydrolysis to fermentable sugars, and the sugars are converted into ethanol by way of fermentation, wherein the milled raw materials are converted into mash in a liquid receptacle, and proteins are washed away prior to starch degradation, followed by solid particles containing most of the starch being separated from the mash, and the protein content is precipitated from the washing liquid by raising the temperature to 50 to 90° C., preferably 60 to 70° C., and lowering the pH to 1.0 to 5.0, preferably 2.5 to 4.5.

The essence of the invention is a method for processing raw vegetable material, especially legumes, and in particular peas, field beans, lentils, soya beans, lupin beans, chickpeas, winged peas, Arachis, pigeon peas, jack beans, earth peas, lablab beans, peavines, winter peas, vetches, serradella, into protein having a nutritional and feed value, bioethanol, biogas and fertiliser materials, consisting in separating the raw material in the form of husks, milling seed residues, extracting the protein to a liquid phase, separating the solid phase and submitting it to enzymatic hydrolysis, microbial fermentation and biogasification, characterised in that the plant material undergoes a dehusking process, and the resulting husks are hydrolysed and biogasified using the CSTR method. The dehusked raw material is milled to a fraction with a size of 0,1 to 3 mm and extracted with stirring for 1 to 12 h at a temperature of 20° to 80° C., pH 6 to 10 in excess extraction buffer in the range of 3 to 15 times the plant material feed by weight. The insoluble solid fraction is separated and subjected to enzymatic hydrolysis using liquefaction and saccharification enzymes, then subjected to ethanol fermentation at a temperature of 25° to 40° C. for 24 to 72 h at a pH of 4 to 8, where the ethanol formed undergoes distillation, and the digestate is transferred as a substrate for biogas production. After the extraction process, the liquid fraction is precipitated for 1 to 24 h at a temperature of 0 to 30° C. at pH 2 to 6 into a solid in the form of a protein precipitate, which is washed with an extraction buffer with a pH of 4 to 8 in a mass ratio of solid fraction : buffer 1:2. The liquid residue is then removed and subjected to a CSTR biogasification process, and the wet precipitate obtained during the biogasi-fication is used as a fertiliser or is formed into dry pellets and used as an organic fertiliser.

Preferably, the solid form in the form of a protein precipitate after the precipitation process is washed several times with an extraction buffer of pH 4 to 8 in a mass ratio of solid fraction: buffer 1:2.

Preferably, the solid form in the form of a protein precipitate after the precipitation process is washed with an aqueous solution with an organic solvent and dried to obtain the dry fraction.

Preferably, the solid form in the form of a protein precipitate after the precipitation process is washed with an aqueous solution with ethanol at a concentration of 30 to 90%, pH 4 to 8, at a mass ratio of solid fraction: solution of 1:2.

Preferably, the solid form in the form of a protein precipitate after the precipitation process is washed with an aqueous solution with 30 to 90% isopropanol at pH 4 to 8 in a mass ratio of solid fraction: solution of 1:2.

The method according to the invention allows for processing raw plant material, especially legumes, into pure forms of protein having a nutritional and feed value while making use of the resulting by-product streams. The method according to the invention is zero-waste, as it is carried out in a closed cycle, where the waste produced in the process is used to produce bioethanol, biogas and fertiliser materials.

### EMBODIMENT I

In the embodiment, the method for processing legumes into protein having a nutritional and feed value, bioethanol, biogas and fertiliser materials involves removing the husks, milling the dehusked seeds, extracting the protein into an aqueous solvent, precipitating the protein, washing the protein with an aqueous solution, washing the protein with a solvent solution, drying the protein, and subjecting the residue to ethanol fermentation and biogasification. The raw material in the form of pulses is subjected to a dehusking process, where the resulting husks are subjected to hydrolysis and biogasification using the CSTR (Continuous Stirred Tank Reactor) method. The CSTR method involves a continuous process of residue biogasification in a bioreactor to which there is added a medium in the form of waste and a consortium of anaerobic bacteria that transform the available carbon into methane (biogas). The dehusked material is mechanically milled to a fraction with a size of 0.1 mm, suspended in extraction buffer pH 9 at a feedstock to buffer ratio of 1:10 by weight and is extracted for 1 h at 40° C. with stirring. The insoluble fraction is separated from the soluble fraction and hydrolysed using enzymes into monosaccharides, followed by ethanol fermentation, where the resulting ethanol is distilled and the digestate is transferred as a substrate for biogas production. In the soluble fraction, the pH is adjusted to 4.0, the temperature is lowered to 4° C., and the precipitation process is carried out for 4 h. The protein precipitate formed during the process is separated from the liquid phase and washed once with water pH 4 in a wet protein : water ratio of 1:2, washed subsequently with a 30% ethanol solution pH 4 in a wet protein : water ratio of 1:2 and dried. The liquid fraction is biogasified by CSTR, and the wet precipitate produced after biogasification is used as a fertiliser or formed into dry pellets and used as an organic fertiliser.

### EMBODIMENT II

In the embodiment, the method for processing legumes into protein having a nutritional and feed value, bioethanol, biogas and fertiliser materials involves removing the husks, milling the dehusked seeds, extracting the protein into an aqueous solvent, precipitating the protein, washing the protein, drying the protein and subjecting the residue to ethanol fermentation and biogasification. The raw material in the form of legumes is subjected to a dehusking process, where the resulting husks are subjected to hydrolysis and biogasification using the CSTR (Continuous Stirred Tank Reactor) method. The CSTR method involves a continuous process of residue biogasification in a bioreactor to which there is added a medium in the form of waste and a consortium of anaerobic bacteria that transform the available carbon into methane (biogas). The dehusked material is mechanically milled to a fraction with a size of 2 mm, suspended in extraction buffer pH 8 at a feedstock to buffer ratio of 1:10 by weight and is extracted for 1 h at 40° C. with stirring. The insoluble fraction is separated from the soluble fraction and hydrolysed using enzymes into monosaccharides, followed by ethanol fermentation, where the resulting ethanol is distilled and the digestate is transferred as a substrate for biogas production. In the soluble fraction, the pH is adjusted 4.0, the temperature is lowered to 4° C., and the precipitation process is carried out for 4 h. The resulting protein precipitate is separated from the liquid phase and washed with water pH 7 in a wet protein : water ratio of 1:2 and dried. The liquid fraction is biogasified by CSTR, and the wet precipitate produced after biogasification is used as a fertiliser or formed into dry pellets and used as an organic fertiliser.

### EMBODIMENT III

In the embodiment, the method for processing legumes into protein having a nutritional and feed value, bioethanol, biogas and fertiliser materials involves removing the husks, milling the dehusked seeds, extracting the protein into an aqueous solvent, precipitating the protein, washing the protein with an aqueous solution, washing the protein with a solvent solution, drying the protein, and subjecting the residue to ethanol fermentation and biogasification. The raw material in the form of pulses is subjected to a dehusking process, where the resulting husks are subjected to hydrolysis and biogasification using the CSTR method. The dehusked material is mechanically milled to a fraction with a size of 0.5 mm, suspended in extraction buffer pH 8 at a feedstock to buffer ratio of 1:15 by weight and is extracted for 12 h at 20° C. with stirring. The insoluble fraction is separated from the soluble fraction and hydrolysed using enzymes into monosaccharides, followed by ethanol fermentation, where the resulting ethanol is distilled and the digestate is transferred as a substrate for biogas production. In the soluble fraction, the pH is adjusted to 5.0, the temperature is lowered to 10° C. and the precipitation process is conducted for 24 h, the protein precipitate formed during the process is separated from the liquid phase and washed twice with water pH 6 in a wet protein : water ratio of 1:3, washed subsequently with a 10% isopropanol solution pH 7 in a wet protein : water ratio of 1: 1 and dried. The liquid fraction is biogasified by CSTR, and the wet precipitate produced after biogasification is used as a fertiliser or formed into dry pellets and used as an organic fertiliser.

### EMBODIMENT IV

In the embodiment, the method for processing legumes into protein having a nutritional and feed value, bioethanol, biogas and fertiliser materials involves removing the husks, milling the dehusked seeds, extracting the protein into an aqueous solvent, precipitating the protein, washing the protein with an aqueous solution, washing the protein with a solvent solution, drying the protein, and subjecting the residue to ethanol fermentation and biogasification. The raw material in the form of pulses is subjected to a dehusking process, where the resulting husks are subjected to hydrolysis and biogasification using the CSTR method. The dehusked material is mechanically milled to a fraction with a size of 3 mm, suspended in extraction buffer pH 6 at a feedstock to buffer ratio of 1: 4 by weight and is extracted for 1 h at 80° C. with stirring. The insoluble fraction is separated from the soluble fraction and hydrolysed using enzymes into monosaccharides, followed by ethanol fermentation, where the resulting ethanol is distilled and the digestate is transferred as a substrate for biogas production. In the soluble fraction, the pH is adjusted to 2.0, the temperature is lowered to 30° C., and the precipitation process is carried out for 12 h. The protein precipitate obtained is separated from the liquid phase and washed four times with water pH 5 in a wet protein : water ratio of 1:5, washed subsequently with a 10% ethanol solution pH 7 in a wet protein : water ratio of 1:2 and dried. The liquid fraction is biogasified by CSTR, and the wet precipitate produced after biogasification is used as a fertiliser or formed into dry pellets and used as an organic fertiliser.

### EMBODIMENT V

In the embodiment, the method for processing legumes into protein having a nutritional and feed value, bioethanol, biogas and fertiliser materials involves removing the husks, milling the dehusked seeds, extracting the protein into an aqueous solvent, precipitating the protein, washing the protein with an aqueous solution, washing the protein with a solvent solution, drying the protein, and subjecting the residue to ethanol fermentation and biogasification. The raw material in the form of pulses is subjected to a dehusking process, where the resulting husks are subjected to hydrolysis and biogasi-fication using the CSTR method. The dehusked material is mechanically milled to a fraction with a size of 0.2 mm, suspended in extraction buffer pH 7 at a feedstock to buffer ratio of 1:10 by weight and is extracted for 1 h at 40° C. with stirring. The insoluble fraction is separated from the soluble fraction and hydrolysed using enzymes into monosaccharides, followed by ethanol fermentation, where the resulting ethanol is distilled and the digestate is transferred as a substrate for biogas production. In the soluble fraction, the pH is adjusted to 5.0, the temperature is lowered to 10° C., and the precipitation process is carried out for 24 h. The resulting protein precipitate is separated from the liquid phase and washed twice with water pH 4 in a wet protein : water ratio of 1:2 and dried. The liquid fraction is biogasified by CSTR, and the wet precipitate produced after biogasification is used as a fertiliser or formed into dry pellets and used as an organic fertiliser.

### EMBODIMENT VI

In the embodiment, the method for processing legumes into protein having a nutritional and feed value, bioethanol, biogas and fertiliser materials involves removing the husks, milling the dehusked seeds, extracting the protein into an aqueous solvent, precipitating the protein, washing the protein with an aqueous solution, washing the protein with a solvent solution, drying the protein, and subjecting the residue to ethanol fermentation and biogasification. The raw material in the form of pulses is subjected to a dehusking process, where the resulting husks are subjected to hydrolysis and biogasification using the CSTR method. The dehusked material is mechanically milled to a fraction with a size of 1 mm, suspended in extraction buffer pH 10 at a feedstock to buffer ratio of 1: 3 by weight and is extracted for 6 h at 50° C. with stirring. The insoluble fraction is separated from the soluble fraction and hydrolysed using enzymes into monosaccharides, followed by ethanol fermentation, where the resulting ethanol is distilled and the digestate is transferred as a substrate for biogas production. In the soluble fraction, the pH is adjusted to 2.0, the temperature is lowered to 0° C. and the precipitation process is conducted for 1 h, the protein precipitate formed during the process is separated from the liquid phase and washed once with water pH 8 in a wet protein : water ratio of 1:5, washed subsequently with a 90% ethanol solution pH 4 in a wet protein : water ratio of 1: 1 and dried. The liquid fraction is biogasified by CSTR, and the wet precipitate produced after biogasification is used as a fertiliser or formed into dry pellets and used as an organic fertiliser.

### EMBODIMENT VII

In the embodiment, the method for processing legumes into protein having a nutritional and feed value, bioethanol, biogas and fertiliser materials involves removing the husks, milling the dehusked seeds, extracting the protein into an aqueous solvent, precipitating the protein, washing the protein with an aqueous solution, washing the protein with a solvent solution, drying the protein, and subjecting the residue to ethanol fermentation and biogasification. The raw material in the form of pulses is subjected to a dehusking process, where the resulting husks are subjected to hydrolysis and biogasi-fication using the CSTR method. The dehusked material is mechanically milled to a fraction with a size of 0,1 mm, suspended in extraction buffer pH 9 at a feedstock to buffer ratio of 1:10 by weight and is extracted for 2 h at 40° C. with stirring. The insoluble fraction is separated from the soluble fraction and hydrolysed using enzymes into monosaccharides, followed by ethanol fermentation, where the resulting ethanol is distilled and the digestate is transferred as a substrate for biogas production. In the soluble fraction, the pH is adjusted to 4.0, the temperature is lowered to 0° C., and the precipitation process is carried out for 24 h. The resulting protein precipitate is separated from the liquid phase and washed with a 40% ethanol solution pH 4 in a wet protein : solution mass ratio 1:2 and dried. The liquid fraction is biogasified by CSTR, and the wet precipitate produced after biogasification is used as a fertiliser or formed into dry pellets and used as an organic fertiliser.

### EMBODIMENT VIII

In the embodiment, the method for processing legumes into protein having a nutritional and feed value, bioethanol, biogas and fertiliser materials involves removing the husks, milling the dehusked seeds, extracting the protein into an aqueous solvent, precipitating the protein, washing the protein with an aqueous solution, washing the protein with a solvent solution, drying the protein, and subjecting the residue to ethanol fermentation and biogasification. The raw material in the form of pulses is subjected to a dehusking process, where the resulting husks are subjected to hydrolysis and biogasification using the CSTR method. The dehusked material is mechanically milled to a fraction with a size of 2 mm, suspended in extraction buffer pH 7 at a feedstock to buffer ratio of 1: 5 by weight and is extracted for 8 h at 20° C. with stirring. The insoluble fraction is separated from the soluble fraction and hydrolysed using enzymes into monosaccharides, followed by ethanol fermentation, where the resulting ethanol is distilled and the digestate is transferred as a substrate for biogas production. In the soluble fraction, the pH is adjusted to 2.0, the temperature is lowered to 10° C., and the precipitation process is carried out for 12 h. The protein precipitate obtained is separated from the liquid phase and washed three times with water pH 5 in a wet protein : water mass ratio of 1:5, washed subsequently with a 90 % isopropanol solution pH 4 in a wet protein : water mass ratio of 1: 2 and dried. The liquid fraction is biogasified by CSTR, and the wet precipitate produced after biogasification is used as a fertiliser or formed into dry pellets and used as an organic fertiliser.

### EMBODIMENT IX

In the embodiment, the method for processing legumes into protein having a nutritional and feed value, bioethanol, biogas and fertiliser materials involves removing the husks, milling the dehusked seeds, extracting the protein into an aqueous solvent, precipitating the protein, washing the protein with an aqueous solution, washing the protein with a solvent solution, drying the protein, and subjecting the residue to ethanol fermentation and biogasification. The raw material in the form of pulses is subjected to a dehusking process, where the resulting husks are subjected to hydrolysis and biogasi-fication using the CSTR method. The dehusked material is mechanically milled to a fraction with a size of 0.1 mm, suspended in extraction buffer pH 10 at a feedstock to buffer ratio of 1: 15 by weight and is extracted for 1 h at 80° C. with stirring. The insoluble fraction is separated from the soluble fraction and hydrolysed using enzymes into monosaccharides, followed by ethanol fermentation, where the resulting ethanol is distilled and the digestate is transferred as a substrate for biogas production. In the soluble fraction, the pH is adjusted to 6.0, the temperature is lowered to 0° C., and the precipitation process is carried out for 1 h. The resulting protein precipitate is separated from the liquid phase and washed with a 60% isopropanol solution pH 8 in a wet protein : solution mass ratio 1:2 and dried. The liquid fraction is biogasified by CSTR, and the wet precipitate produced after biogasification is used as a fertiliser or formed into dry pellets and used as an organic fertiliser.

### EMBODIMENT X

In the embodiment, the method for processing legumes into protein having a nutritional and feed value, bioethanol, biogas and fertiliser materials involves removing the husks, milling the dehusked seeds, extracting the protein into an aqueous solvent, precipitating the protein, washing the protein with an aqueous solution, washing the protein with a solvent solution, drying the protein, and subjecting the residue to ethanol fermentation and biogasification. The raw material in the form of pulses is subjected to a dehusking process, where the resulting husks are subjected to hydrolysis and biogasi-fication using the CSTR method. The dehusked material is mechanically milled to a fraction with a size of 2 mm, suspended in extraction buffer pH 8 at a feedstock to buffer ratio of 1: 8 by weight and is extracted for 2 h at 30° C. with stirring. The insoluble fraction is separated from the soluble fraction and hydrolysed using enzymes into monosaccharides, followed by ethanol fermentation, where the resulting ethanol is distilled and the digestate is transferred as a substrate for biogas production. In the soluble fraction, the pH is adjusted to 3.0, the temperature is lowered to 20° C., and the precipitation process is carried out for 8 h. The protein precipitate obtained is separated from the liquid phase and washed three times with water pH 5 in a wet protein : water mass ratio of 1:3, washed subsequently with a 40% isopropanol solution pH 5 in a wet protein : water mass ratio of 1: 2 and dried. The liquid fraction is biogasified by CSTR, and the wet precipitate produced after biogasification is used as a fertiliser or formed into dry pellets and used as an organic fertiliser.

1. Method of processing raw plant material, especially legumes into protein having a nutritional and feed value, bioethanol, biogas and fertiliser materials, consisting in separating raw material in the form of husks, grinding the seed residues, extracting protein to a liquid phase, separating the solid phase and subjecting it to enzymatic hydrolysis, microbial fermentation and biogasification, characterised in that the raw plant material is dehusked, the resulting husks are subjected to hydrolysis and biogasification using the CSTR method, followed by the dehusked material being milled to a fraction with a size of 0.1 to 3 mm and extracted with stirring for 1 to 12 h at 20 to 80° C., at pH 6 to 10 in excess extraction buffer at a range of 3 to 15 times the weight of the plant feedstock, wherein the insoluble solid fraction is separated and subjected to enzymatic hydrolysis with liquefying and saccharifying enzymes, followed by ethanol fermentation at a temperature of 25 to 40° C. for 24 to 72 h at pH 4 to 8, where the produced ethanol is distilled and the digestate is transferred as a substrate for biogas production, and the liquid fraction after the extraction process is subjected to a process of precipitation for 1 to 24 h at a temperature of 0 to 30° C., pH 2 to 6 into a solid in the form of a protein precipitate, which is washed with an extraction buffer with a pH 4 to 8 in a solid fraction : buffer mass ratio of 1:2, after which the liquid residue is removed, which is subjected to a biogasification process using the CSTR method, and the wet precipitate produced after biogasification is used as a fertiliser or formed into dry pellets and used as an organic fertiliser.

2. The method according to claim 1 characterised in that the solid form in the form of a protein sludge after the precipitation process is washed several times with an extraction buffer of pH 4 to 8 in a mass ratio of solid fraction: buffer 1: 2.

3. The method according to claim 1 characterised in that the solid form in the form of a protein sludge after the precipitation process is washed with an aqueous solution with an organic solvent and dried to obtain the dry fraction.

4. The method according to claim 3 characterised in that the solid form in the form of a protein sludge after the precipitation process is washed with an aqueous solution with ethanol at a concentration of 30 to 90%, pH 4 to 8, at a mass ratio of solid fraction: solution of 1: 2.

5. The method according to claim 1 characterised in that the solid form in the form of a protein sludge after the precipitation process is washed with an aqueous solution with 30 to 90% isopropanol at pH 4 to 8 in a mass ratio of solid fraction: solution of 1: 2.
